(19) United States
(12) Patent Application Publication
(10) Pub. No.: US 2015/0012066 A1
(43) Pub. Date: Jan. 8, 2015

(54) NONINVASIVE DELIVERY AND CONTROL OF STIMULATION SIGNALS

(71) Applicant: Wendell Martin Underwood, Tampa, FL (US)

(72) Inventor: Wendell Martin Underwood, Tampa, FL (US)

(21) Appl. No.: 14/492,339

(22) Filed: Sep. 22, 2014

Related U.S. Application Data
(63) Continuation of application No. PCT/US2013/033490, filed on Mar. 22, 2013.
Application No. PCT/US2013/033490 is a continuation-in-part of application No. 13/427,000, filed on Mar. 22, 2012.

(51) Int. Cl. A61N 1/36; A61N 1/04; A61N 1/372

(52) U.S. Cl. CPC A61N 1/36014; A61N 1/37235; A61N 1/37217; A61N 1/0492
USPC 607/59

(57) ABSTRACT

System and method for the non-invasive, wireless generation, delivery, and control of electrostimulation signals. The system contains a circuit board with a plurality of components protected by a silicone housing disposed in overlying relation to form a hollow interior therein. Collectively, this is one electrode. A plurality of electrodes are attached to an individual's body via leads on the underside of the electrodes. The electrodes are structured and adapted to transmit an electrical impulse into the individual's body, thereby performing a treatment (e.g., for pain relief). An electronic device can be used to activate or deactivate each electrode, along with control the length and strength of impulses, depending on the protocol used for the individual's treatment. Each impulse is made up of a plurality of bursts, and each burst is made up of a plurality of pulses, which are higher voltage signals transmitted from the circuit board through the leads.

### CROSS REFERENCE TO RELATED APPLICATION

This nonprovisional application is a continuation of and claims priority to prior filed International Application PCT/US2013/033490 filed Mar. 22, 2013, and claims priority to currently pending LLS. Nonprovisional patent application Ser. No. 13/427,000, entitled “NEEDLE-LESS/WIRELESS ACUPUNCTURE SYSTEM AND METHOD,” filed on Mar. 22, 2012, the contents of which are hereby incorporated by reference,

### BACKGROUND OF THE INVENTION

1. Field of the Invention

This invention relates, generally, to acupuncture therapy. More particularly, it relates to a noninvasive and wireless system and method for performing acupuncture therapy on a subject.

2. Description of the Prior Art

Acupuncture is a well-known alternative medicine methodology that aids in pain relief and other treatments to retain homeostasis within an individual's body. Acupoints are believed in position to stimulate the central nervous system to release certain chemicals (e.g., hormones) into the central nervous system and other muscles. These chemicals may enhance the individual's body's ability to heal itself. Experts predict various mechanisms of the effects of acupuncture procedure. Some believe acupoints conduct electromagnetic signals, such that stimulating acupoints enhance the body's ability to relay electromagnet signals to initiate or increase the flow of the body's natural pain-killing biochemicals (e.g., endorphins) or immune system cells. Others predict that stimulation of acupoints activate opioid systems and permit the release of opioids into the central nervous system, thereby alleviating pain in the individual. Even others predict that acupuncture can alter brain chemistry (i.e., release of different neurotransmitters), body sensation, and involuntary body functions (e.g., regulatory actions). In short, acupuncture has become a mainstream method for an individual to treat his/her pain or other physical or mental issues.

During a typical acupuncture procedure, an array of thin needles are inserted into the acupuncture points of a patient's body. Because acupuncture involves the penetration of skin, it is an invasive procedure that consequently has several risk factors and possible complications. This is particularly true if the acupuncture procedure is not performed by a well-trained licensed practitioner. Risks of acupuncture include, but are not limited to, injury to vital nerves and structures, bleeding, passage of infections if needles are not properly sterilized, bruising, dizziness, fainting, increased pain, nausea, among others. The primary cause for each of these risks is the need to use needles and puncturing of the skin.

Attempts have been made at noninvasive acupuncture that does not involve the puncturing of the skin. However, these methodologies include acupressure, pushback pins on acupuncture guns that touch the skin but do not penetrate, stimulation of acupuncture points using laser or electrical stimulation, etc. Additionally, only up to four (4) acupoints can be stimulated at once. Thus, these alternative methodologies as seen in the prior art are not as effective and efficient as standard acupuncture methodologies.

Additionally, the wires required for the electrical and laser stimulation disrupt the therapy room. The wires hinder the practitioner's ability to complete the procedure safely, effectively, and efficiently. Also, use of wires may cause fluctuation in the stimulation applied to the acupuncture point, as the wires may move. This can also create monitoring errors (i.e., artifact) caused by crossing of wires. Wires also increase the risks of the pulling or tugging on the structures attached to the patient's body and wrapping around the patient's extremities or getting caught on structures in the external environment. Separate packaging fir wires also is more expensive in manufacture.

Few wireless transcutaneous electric nerve stimulation (TENS) units do exist in the conventional art, including the HOLLYWOG WiTouch Wireless TENS Unit, Free-Q Wireless TENS Unit, and IBP Wireless Mini TENS Unit, which are incorporated herein by reference. Additionally, iHealth has integrated self-monitoring health/medical devices (e.g., digital scales, blood pressure monitors) with mobile technology, which also is incorporated herein by reference. However, the conventional art poses several issues as well, for example the inflexibility of delivering set frequencies at set wavelengths and the limited range of electrical impulses deliverable to an individual.

Accordingly, what is needed is a pain treatment system and method that maximizes the benefits of acupuncture, effectiveness, efficiency, ease-of-use, and accuracy, but also minimizes the risks involved. However, in view of the art considered as a whole at the time the present invention was made, it was not obvious to those of ordinary skill how the art could be advanced.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable staturory provisions; or is known to be relevant to an attempt to solve any problem with which this specification is concerned.

SUMMARY OF THE INVENTION

The long-standing but heretofore unfulfilled need for an improved pain treatment system and methodology is now met by a new, useful and nonobvious invention.

In an embodiment, the current invention comprises a wireless electrostimulation system for generating and controlling stimulation signals delivered to the skin of an individual, The system includes electrodes each having a housing with a contact portion and a non-contact portion, forming a closed hollow interior. The contact portion is planar and has an inner surface (facing the interior) and an outer surface (facing the exterior). A circuit board forms the contact portion entirely or is disposed on the contact portion within the interior of the housing. The circuit board has a battery and several electrical components that would, in part, receive an incoming electrical signal containing data and deliver an outgoing electrical signal in the form of an electrical impulse. Two of the components can be a controller component and a stimulator component. The controller component manages the operation of the components, and the stimulator component generates high voltage charges that are delivered within the electrical impulses (e.g., as pulses). Each electrical pulse is formed of several bursts, and each burst is formed of several pulses. Each burst has a predetermined interval; each pulse has a predetermined waveform, voltage level, and interval. These are the parameters that are contained in the data of the incoming electrical signal and thus can be adjusted prior to or during treatment of the individual. The components further include several low voltage signals that pass between the controller and stimulator components. These signals monitor and control the outgoing electrical signal and thus what is experienced by the individual during treatment. Additionally, there are electrical leads placed on the contact portion on the opposite side of the electrical components, and a conductive adhesive gel is placed over top the electrical leads. This allows the electrodes to adhere to the individual's skin and receive the electrical impulses from the circuit board. As a separate aspect of the whole system, an electronic device (e.g., smartphone, dedicated device, computer, tablet, etc.) is used to wirelessly transmit the incoming electrical signal from the device to the electrodes.

An activation push button or switch can be positioned within the interior of the housing for activating the electrode when the housing is depressed or switch is toggled.

The low voltage signals may include a stimulating signal, a pulse width modulated signal, and/or a variation potential signal. The stimulating signal is generated by the controller component and notifies the stimulator component when the discharge the outgoing electrical signal, or electrical impulse. The pulse width modulated signal is generated by the controller component and controls whether the voltage within each burst is increased or decreased. The variation potential signal is generated by the stimulator component and lets the pulse width modulated signal know whether to produce more or less of its signal.

The parameters or settings set within the electronic device and transmitted to each electrode could be in the form of an exact acupuncture protocol, depending on the needs of the individual undergoing treatment.

The electronic device may include a master component, and the components on the circuit board may include a slave component. The master and slave components would be in wireless, electrical communication with each other to monitor and control the outgoing electrical signal, or electrical impulse. In a further embodiment, this wireless, electrical communication can be a BLUETOOTH connection between the master and slave components.

In a separate embodiment, the current invention comprises a method for controlling delivery of stimulation signals to an individual in need. Electrodes are provided with a housing, a circuit board imbedded within the housing, electrical leads on the exterior of the housing, and conducting adhesive gel disposed in overlying relation to the electrical leads. Each electrode is capable of receiving an incoming electrical signal and transmitting an outgoing electrical signal that becomes an electrical impulse delivered to the individual in treatment. Each electrical impulse is formed of several bursts, and each burst is formed of several pulses. An electronic device is provided and can monitor and control the electrodes and their output. The individual is evaluated to determine what treatment is needed for him/her, and based on this treatment, a treatment protocol can be derived and set on the electronic device. The treatment protocol includes the waveform of each pulse, voltage level of each pulse, interval of each pulse, and/or interval of each burst. The treatment protocol is included in the incoming electrical signal, as the electronic device wirelessly transmits the incoming electrical signal to the circuit board. The electrodes are then attached to the skin of the individual in treatment by contacting the skin with the conducting adhesive gel and leads. Each electrode is activated to initiate the treatment and discharge the electrical impulse. Each burse and pulse are monitored and controlled by the electronic device and the above-referenced parameters can be adjusted as needed.

When the individual is evaluated and type of treatment is selected, the treatment protocol may be determined automatically by software installed on the electronic device. The electronic device can then automatically wirelessly transmit the parameters of the treatment protocol to the electrodes.

An activation button or switch may be positioned within the interior of the housing. Thus, the electrode can be activated by manually pushing the button or toggling the switch.

The components of the circuit board may include a controller component and a stimulator component that are in electrical communication with each other. in a further embodiment, the electrical communication between the two components can be low voltage signals, such as a stimulating signal, a pulse width modulated signal, and/or a variation potential signal. The stimulating signal is generated by the controller component and notifies the stimulator component when the discharge the outgoing electrical signal, or electrical impulse. The pulse width modulated signal is generated by the controller component and controls whether the voltage within each burst is increased or decreased. The variation potential signal is generated by the stimulator component and lets the pulse width modulated signal know whether to produce more or less of its signal.

The treatment protocol may correspond directly to an acupuncture protocol, as needed by the individual in treatment.

The electronic device may include a master component, and the electrode may include a slave component on its circuit board. The master and slave components are in wireless, electrical communication with each other to monitor and control the outgoing electrical signals (i.e., the electrical impulses). In a further embodiment, there may be a wireless BLUETOOTH connection between the master and slave components for transmission of signals/data.

In a separate embodiment, the current invention comprises an electrode system for generating and delivering stimulation signals to an individual in need. The system includes a circuit board having a controller component and a stimulator component. A flexible housing is fitted snugly over top of the circuit board to secure/protect the components of the circuit board and to forma hollow interior between the housing and circuit board. A push button is positioned on the circuit board and is in electrical communication with the stimulator component to activate transmission of the stimulation signals to the individual. A battery is disposed in overlying relation to the push button, so that when the housing is depressed/pushed, a force is applied to the battery, which, in turn, depressed the push button, thus activating the electrode system. Copper contacts are positioned in underlying relation to the circuit board, and a conductive adhesive gel is disposed in housing relation to the copper contacts. The gel allows attachment of each electrode to the individual and conducts stimulation signals from the circuit board to the individual.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the nature and objects of the invention, reference should be made to the following detailed disclosure, taken in connection with the accompanying drawings, in which:

FIG. 1 depicts the general structural components of an embodiment of the current invention.

FIG. 2 is a schematic of the printed circuit board contained within the interior of an embodiment of the current invention.

FIG. 3 depicts a pattern including a series of bursts of pulses as might be outputted by an embodiment of the current invention.

FIG. 4 depicts a pattern including a set of pulses as contained within each burst outputted by an embodiment of the current invention.

FIG. 5A is a schematic of the electrical circuit showing voltage supply to the controller and stimulator with voltage signals passing between the controller and stimulator.

FIG. 5B is a schematic of the electrical circuit showing the power between the battery and power leads and showing the individual interactions among the voltage signals.

FIG. 5C is a schematic of the electrical circuits within an embodiment of the current invention.

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

The bio-electricity system, apparatus and methodology of the current invention can be utilized for pain relief, reduction of inflammation, decreasing muscle spasms, and reduction of edema. The stimulation imparted by said system and apparatus includes any known non-invasive stimulation that penetrates the targeted musculature of the subject. For example, this stimulation can include electrical muscle stimulation, interferential current, microcurrent, Russian stimulation, transcutaneous electric nerve stimulation (TENS), among others. The ultimate goal is to treat a subject in need by stimulating appropriate body points by creating bioelectricity, thereby stimulating conductance of nerve pathways for allowing the subject to restore homeostasis—goals commensurate with acupuncture or other non-drug pain therapies. For example, these specific body points can be those found in traditional acupuncture protocol, though other body points can be stimulated as well, depending on the subject. For example, trigger points in the upper back/lower neck region can be stimulated wirelessly for pain relief in that area of the subject's body. This allows the user or subject to selected different types and levels of current and area to be treated.

Generally, the apparatus of the current invention can include one or more Probes/electrodes, adhesives disposed in underlying relation to the probes/electrodes, and specific electronic components within the probes/electrodes. As part of the overall system, a mobile device, such as a smartphone, tablet, computer, or dedicated remote control, is included with a software application to remotely and wirelessly control (i.e., activate, deactivate, regulate, monitor, etc.) the output of the electronic components contained within the probes/electrodes, which are positioned on a user in need thereof. The software application would be accessible from a non-transitory, computer-readable media and provide instructions for a computer processor to wirelessly control the apparatus of the current invention as a function of the malady to be treated.

Structurally, each probe has a planar, circular or quadrangle contact surface with a predetermined diameter or dimensions. A generally hemispherical or cubic, non-contact surface extends in overlying relation to the upper side of the contact surface. If the contact surface is circular, the non-contact surface would have a predetermined diameter that is substantially similar to the predetermined diameter of the contact surface, such that the two surfaces are substantially aligned to form a dome. The dome may have any diameter that would be suitable for operation. In an embodiment, the dome would have a diameter between about 0.4 inches to about 0.6 inches, preferably around 0.5 inches. The dome may have any height that would be suitable for operation. In an embodiment, the dome would have a height between about 0.1 inches and about 0.3 inches, preferably between about 0.125 inches and about 0.25 inches. Typically, in these embodiments, the diameter is between about two (2) and about four (4) times the height.

If the contact surface is quadrangle is shape, the non-contact surface would have predetermined dimensions that are substantially similar to the predetermined dimensions of the contact surface, such that the two surfaces simply form two overlapping layers or even a cube shape if needed. The overlapping layers can have any set of dimensions that would be suitable for operation. In an embodiment, the dimensions can be between about 0.5 and 2 inches by about 1 and 2.5 inches. In a further embodiment, the dimensions are about 1.88 inches by about 2.13 inches. However, these dimensions can be further minimized, up to a 50% reduction in size, by restructuring the electronic component portion (e.g., microcontroller) within the probes by having it run at lower speeds, eliminating connectors, and/or soldering the microcontroller directly to the back of the printed circuit (“PC”) board. The specific electronic components of the current invention will become apparent as this specification continues.

A substantially planar layer of disposable or reusable conducting adhesive is disposed in underlying relation to the contact surface on the lower side of the contact surface (i.e., opposite the non-contact surface). Using this adhesive, each probe is capable of being adhesively secured to a specified area of the body of the subject. An electrical lead is disposed between the contact surface and the adhesive to facilitate transmission of the electrical impulse from the probe to the body of the individual in treatment.

Electronic components are imbedded within each probe and are adapted to generate an electric charge when activated. The current outputted through the electric charge can be increased or decreased based on need of the subject via the mobile control device. The electronic components are adapted to receive wireless activation and control signals from the mobile device in order to activate, deactivate, or control the electric charge for a predetermined amount of time in response to the wireless activation and control signals.

The mobile device is adapted—by incorporation of a software application to function as a master in transmitting wireless activation, inactivation, and control signals to the electronic components functioning as slaves. The software application can be programmed by a user to transmit specific signals wirelessly as a function of the probes positioned on the subject or user and the malady to be treated.

In an embodiment, the current invention further includes a method for treating a malady of a subject who is physically out-of-balance or otherwise in pain. The goal of the current method is to include stimulation of proper body points of the subject by creating bioelectricity, thus stimulating conductance of nerve pathways for allowing the user to reach homeostasis or pain relief. The method includes utilizing the apparatus and software application of the current invention on a subject in need thereof.

The software application includes use of any known wireless technology, for example WI-FI or BLUETOOTH technology, for operating the slave electronic components within the probes in response to the master software application in the mobile device. The software application should be operable both in locations with WI-FI accessibility and in locations without WI-FI accessibility. The software application includes an operating program adapted to activate, deactivate, and control the probes wirelessly as a function of the subject and malady being treated. Because of this wireless technology, in particular BLUETOOTH technology, an array of acupoint sites can be stimulated without inserting a needle.

in an embodiment, the software application can include two (2) main functions: (1) a BLUETOOTH-based controlling module used to send commands to the wireless electrodes, and (2) a user interface subsystem. The user interface subsystem can have a primary purpose of being a help system that uses instructional videos directing a user/operator in electrode/pad placement based on the desired treatment plan. Additionally, the user interface of the software application can present two controls per pad/electrode that regular the pulse intensity and frequency, along with a timer feature that controls duration of the treatment. This software application also contemplates the abilities to review and record treatment history, as well as web-based access. The software application is adapted to run on any platform (e.g., ANDROID, Apple iOS, etc.) and is accessible from anon-transitory, computer-readable medium.

The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire-line, optical fiber cable, radio frequency, etc., or any suitable combination of the foregoing. Computer program code for carrying out operations for aspects of the present invention may be written in any combination of one or more programming languages, including an object oriented programming language such as Java, C#, C++ or the like and conventional procedural programming languages, such as the “C” programming language or similar programming languages.

The computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified.

### Example 1

In an embodiment, a plurality of probes/electrodes are applied to multiple acupressure points. Each acupoint is stimulated by an electrical impulse through the respective probe attached to that acupoint. BLUETOOTH technology is utilized to deliver the impulse from a software application containing a computer processor to said respective probe.

The probe is not set to a specific frequency unit and delivers an impulse into the tissue that will stimulate that particular acupoint and/or trigger point. More specifically, the electrical impulse is not delivered at a frequency and wavelength of a TENS unit. Impulse strength can be increased or decreased individually (i.e., within each probe) or collectively (i.e., all probes).

The electrical impulses are delivered at a set protocol/pattern as taught by acupuncture techniques. Acupuncture teaches where to put the probes, whereas the prior art teaches the placement of probes on the specific points of pain and thus teach away from the current invention. The protocol is preset, rather than being flexible based on pain experienced by the individual as taught in the conventional art. The probes of the current invention differ from the prior art mainly in how the electrical impulses are delivered and in where the electrical impulses are delivered.

Any acupuncture protocol can be used, depending on the type of therapy required by the individual and the areas of treatment. For example, acupuncture protocols may differ based its purpose, such as pain relief, allergies, to stop smoking, weight loss, fertility treatments, etc. However, this embodiment of the current invention would follow one of these known, preset protocols to provide the appropriate stimulation on certain areas of the individual's body. For instance, if an individual desires acupuncture for treatment of behavioral health (e.g., addictions, mental health, etc.), that individual may follow the protocol spelled out by the National Acupuncture Detoxification Association.

### Example 2

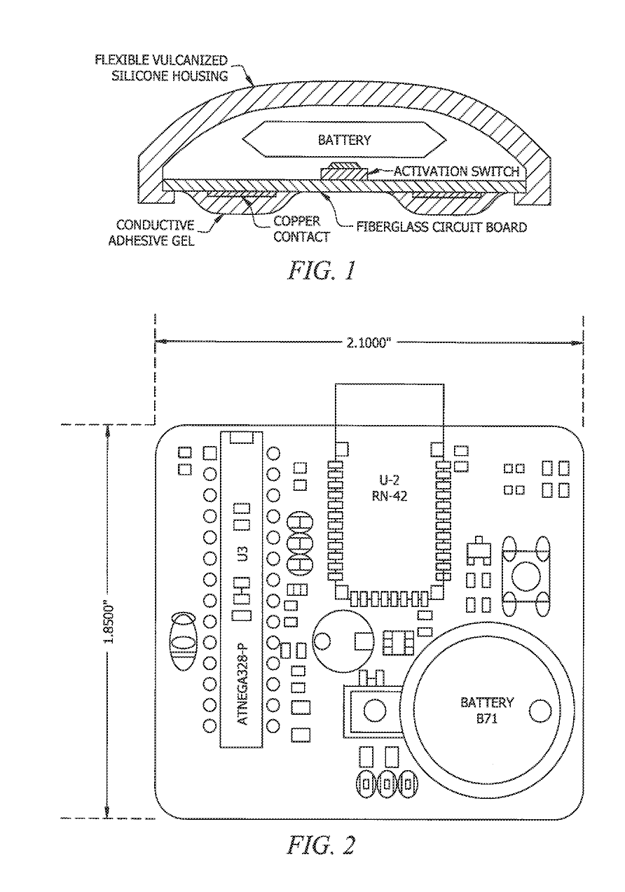

The structure of an embodiment of the current invention is shown in FIG. 1, and the internal circuit board of each probe is depicted in FIG. 2. The wireless stimulation pad or probe is a small, battery-powered device that attaches or is otherwise secured to the skin of a user and delivers therapeutic electrical pulses through the tissue of the user. The device is controlled with a wireless BLUETOOTH interface that permits adjustment of the pulse amplitude, pulse rate, pulse duration, and overall length of treatment. The pulse is delivered from the device, through copper pads on the board, to electro-conductive gel that is adhered to the skin of the individual.

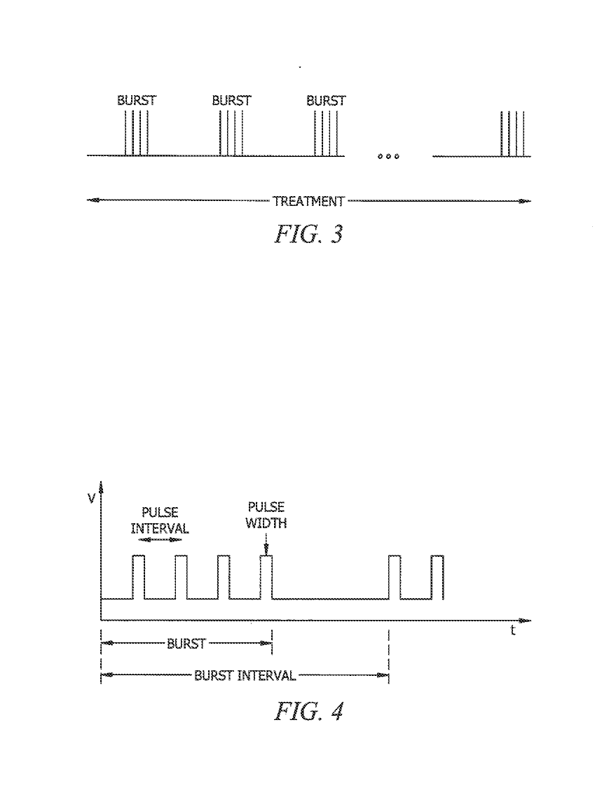

A treatment can last from five (5) to sixty (60) minutes and include a series of bursts of pulses, as depicted in FIG. 3. Each burst includes a set of pulses of electrical voltage across the pad or probe. The voltage is administered to the pad for a pulse width and repeat at regular pulse interval, as depicted in FIG. 4.

As seen in FIGS. 1-2, this embodiment of the current invention contains a plurality of specifications, including, but not limited to a single channel stimulator (e.g., two lead wires or copper pads), a single push button for activation of the electrical circuit, settings adjusted through a BLUETOOTH-enabled device (e.g., smartphone, tablet, computer), and pre-installed software on the BLUETOOTH-enabled device that provides an option to automatically set or adjust each of the foregoing parameters to one of several pre-programmed treatments. These adjustable, controllable settings include waveform (asymmetrical biphasic square pulse), predetermined treatment length (5-60 minutes, adjustable), predetermined pulse voltage (0-60 VDC, adjustable), predetermined pulse width (10-250 microseconds, adjustable), predetermined pulse interval (5-500 milliseconds, adjustable), predetermined burst (0.5-2 seconds, adjustable), and predetermined burst interval (2-10 seconds, adjustable),

More specifically, the single channel stimulator has leads or pads used to transfer the electrical energy received from the device to the tissue of the individual underneath the skin where the pad was applied. The push button located within the interior of the device is used to close the electrical circuit of the pad and supply a voltage to the skin of the individual, as determined by the BLUETOOTH-enabled device, which can control the amount, duration, and interval period of power delivered from the pad to the individual,

The predetermined treatment length indicates the total amount of time for a single session of a particular treatment for an individual in need thereof. During this treatment length, various burst containing a plurality of pulses can be outputted by the electrode either in regular intervals (FIG. 3) or irregular intervals, depending on the needs of the user. Each pulse within each burst can have its own predetermined pulse voltage (FIG. 4), though typically a single burst would include pulses having the same voltage, and predetermined length (or “width”) (FIG. 4), though typically a single burst would include pulses having the same length.

The time from the start of a pulse within a burst to the start of the next pulse is known as the pulse interval and can be adjustable based on how rapid an individual needs the pulses to be administered within each burst. This determines frequency of the pulses within each burst. Between each pulse, there is typically a rest period, where the pulse voltage has been reduced to a lower voltage level or has been turned off completely until the subsequent pulse. Essentially, the waveform of each pulse can be adjusted based on need; examples include sine waves, asymmetric biphasic square, etc.).

The length of a burst itself is the sum of all the pulse intervals within that burst. After a burst has been completed, there is typically a rest period, where the pulse voltage has been reduced to a lower voltage level or has been turned off completely until the subsequent burst. At the point when the next burst is initiated, a burst interval has been completed from the beginning of the previous burst (FIG. 4). This determines frequency of the bursts within the treatment.

To adjust each of the foregoing parameters individually or collectively, a software application can be installed onto the BLUETOOTH-enabled device to control the operations of the entire electrode.

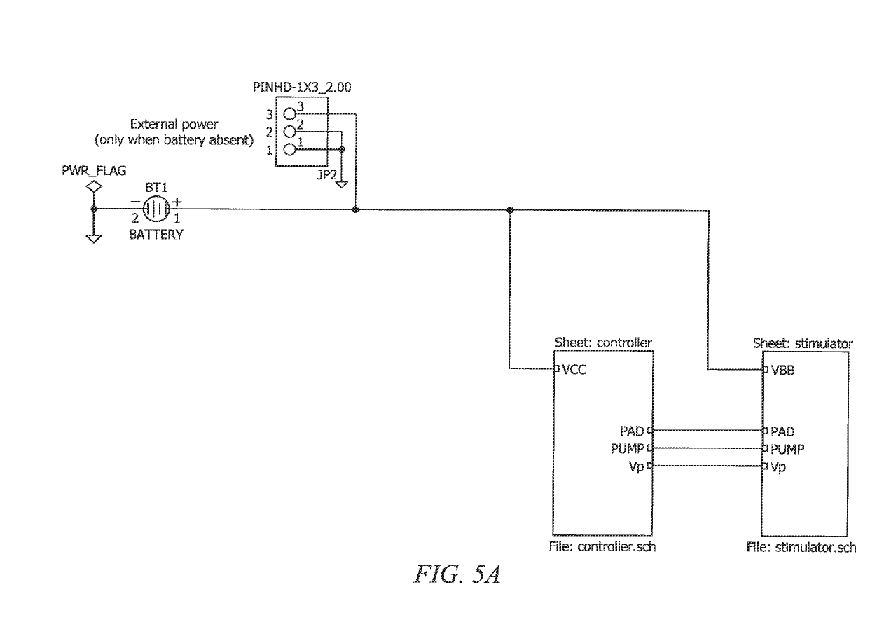

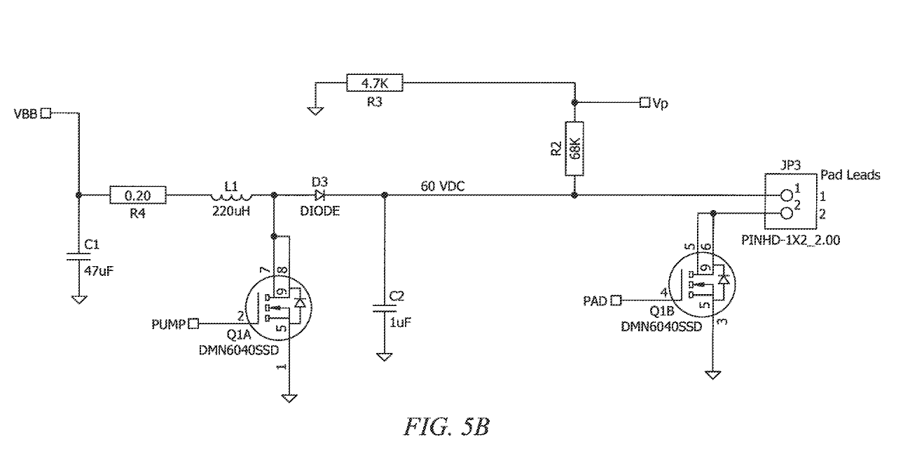

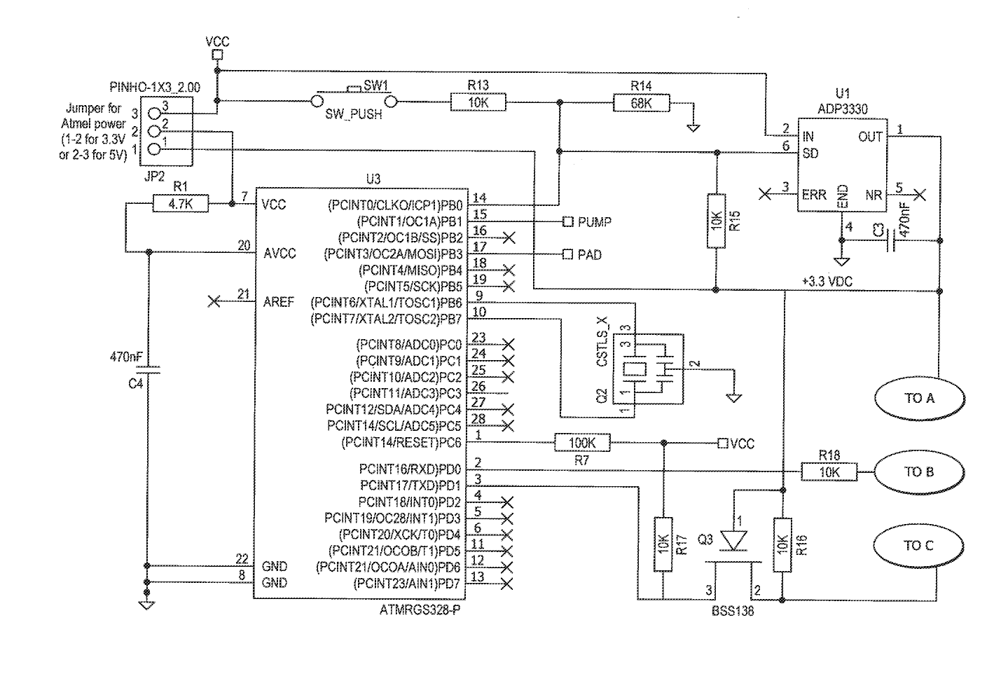

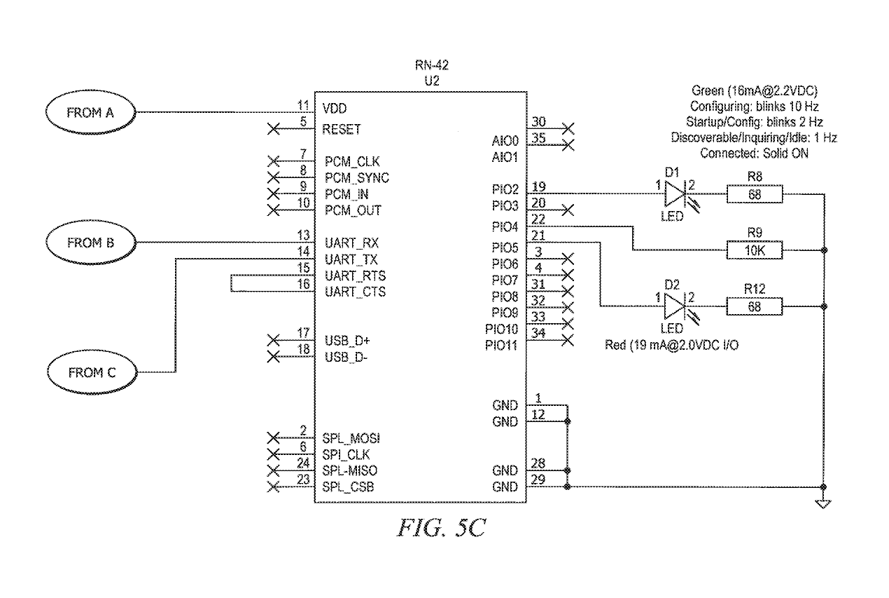

FIGS. 5A-5C are schematics showing these basic functional elements of the electrical probe or pad.

in operation, the device can be broken down into two functional elements: the controller and the stimulator. Each of these functional elements is supplied voltage from a 4-6 VDC coin cell battery or external power supply, as seen in FIG. 5A. The controller manages the overall operation of the device including the pulses and BLUETOOTH communication. The stimulator generates the high voltage charge that is delivered as electrical pulses to electrodes that are in contact with the individual in treatment.

As can be seen in FIGS. 5A (generally) and 5B (more specifically), three low voltage signals can pass between the controller and stimulator: PAD, PUMP, and variation potential (Vp). PAD is the signal from the controller to the stimulator that notifies that stimulator of when the charge is to be delivered to the electrodes. PUMP is a pulse width modulated (PWM) signal generated by the controller that is used by the stimulator in a charge pump circuit. The PWM waveform consists of a fixed-width high (on-state) component and a variable with low (off-state) component that is adjusted by the controller. The Vp signal is a measurement from the stimulator charge-pump capacitor and sent to the controller. When the controller detects that the Vp voltage is lower than what has been set by the user, the PWM waveform is increased in frequency shorter low-states) to speed up the charge pump and raise the voltage. Thus, the controller performs a closed-loop control the charge pump with output PUMP and feedback Vp to achieve a regulated voltage.

From a mechanical perspective, the mechanical design includes a single fiberglass (or other suitable material) circuit board that contains the functional elements described previously. FIG. 1 depicts an embodiment of the structure and organization of the main mechanical components (most of the electronic components are excluded for clarity but can be seen in FIG. 2 with functionality presented in FIGS. 5A-5C). A flexible vulcanized silicone housing will be fitted over the top of the board to protect the electronic components from the outside environment while being comfortable to the wearer. The battery will also be contained within the housing and positioned over the activation switch. When the wearer presses the top of the device, the housing will deflect slightly, applying a force to the battery which, in turn, depresses the activation switch. Thus, the device can be activated and controlled without any external connections or switches. Two copper contacts on the bottom of the board will be where the stimulation pulses are output. Conductive adhesive gel is applied to the bottom of the board over top of the copper contacts to conduct the pulses from the board to the skin of the wearer.

A typical therapy system would include a plurality of these electrodes with pads and circuit boards, along with an electronic device that can monitor or control the parameters of the components contained on the circuit boards. The individual in need should first be evaluated for the type of treatment needed to alleviate the issues or problems experienced by the individual. Once the treatment is specified, a protocol can be determined. Based on this determination of the protocol, a variety of parameters can be set or controlled by the electronic device. The parameters include one or more of the following: waveform of the pulses, overall treatment length, voltage of the pulses, length or width of the pulses, interval of the pulses, length or width of the bursts, and interval of the bursts. These parameters or settings can be set manually by an operator or set automatically by the software application on the electronic device when the type of treatment is determined,

Once these parameters/settings are set in the electronic device, the device sends a wireless signal (e.g., through BLUETOOTH) to the electrodes, which each contain a circuit board with components that can receive said wireless signal containing the set parameters.

The electrodes may be disposable or reusable. If disposable, the wireless signal and data included therein can be stored within an electrical storage component within each electrode. Then a new set of electrodes can be used for a subsequent treatment or within the same treatment. Alternatively, the electrical signal can be carried out in real-time as electrical impulses are outputted through the pads based on these parameters.

If the electrodes are reusable, the wireless signal and data included therein can be stored within an electrical storage component within each electrode. The data can be automatically deleted upon completion of the treatment, whose duration forms a part of that data. Thus, when the treatment terminates, the data is automatically deleted, and the storage component is capable of receiving further data. Alternatively, the data can be stored in the storage component until a new wireless signal with new data is transmitted to the electrode from the electronic device. Another option is that the electrical signal can be carried out in real-time as electrical impulses are outputted through the pads based on these parameters.

in any case, prior to, during, or after transmission of this parameters/settings data, the electrodes are attached to the skin of the individual's body and are positioned according to the treatment protocol determined previously. The electrodes are attached by contacting the individual's skin with the single channel stimulator (e.g., two lead wires or copper pads) on the planar face of the electrode through the conductive adhesive gel covering each lead/pad.

Upon attachment of each electrode to the individual's body, each electrode is activated either by the electronic device (wireless activation) or by pressing the push button within each electrode (manual activation), depending on the structure of the electrodes used.

When the electrodes are activated, the treatment session is initiated. The treatment protocol follows the parameters/settings (e.g., treatment duration, voltage, pulse length, frequency, burst interval, etc.) determined previously. Electrical signals are delivered wirelessly through the leads/pads and through the conductive gel that adheres to the individual's skin. The electrical signals are transmitted to the tissue of the individual through the skin of the individual. If needed, the parameters/settings listed previously may be adjusted in real-time within a treatment, for example if the individual is having an adverse reaction within treatment. Upon completion or termination of the treatment, the electrodes are deactivated via the electronic device (wireless deactivation) or the push button (manual deactivation). Thereafter, the electrodes are removed from the individual's body and disposed of or reused upon sterilization.

Additionally, the current invention contemplates additional benefits of improving usability and lowering cost of manufacture. The coin cell battery can be replaced with a lithium polymer flat-pack battery that can be rechargeable via universal serial bus or other 5 VDC connections. The controller design, as seen in FIG. 5A, can be redesigned around a TEXAS INSTRUMENTS CC2541 that can combine the functionality of the RN-42 and the ATMEL-3298 but in a smaller package. Further, the FR-4 circuit board material can be upgraded to thinner and lighter material, as desired, and can be printed in panels of 6-2.4 units each to reduce manufacturing cost. IR soldering methods can also be utilized to speed up production and reduce failure rate.

The BLUETOOTH antenna can be incorporated into each electrode either as a daughterboard or on the circuit board itself in order to reduce cost and simplify assembly.

The electrical leads can be wired pads covered by the conductive adhesive gel, as described previously. Alternatively, the electrical leads can be replaceable conductive gels, which combine the lead and gel in one component. The replaceable conductive gel also allows a user/operator to reuse electrodes through various treatments.

Costs of using embodiments of the current invention can be further reduced by adding a current measurement circuit and PWM waveforem optimizaiton software inside the electrode itself This allows the device to automatically optimize its settings to reduce power costs.

Costs can be further reduced by running the device on 3.3 VDC to extend the operational time further down the battery discharge curve

Definitions of Claim Terms

Acupuncture protocol: This term is used herein to refer to the settings and parameters of an acupuncture treatment as determined for the needs of an individual.

Burst: This term is used herein to refer to a collection of electrical pulses lasting a predetermined amount of time between the first and last pulse within the collection.

Component: This term is used herein to refer to electronic parts mounted and assembled onto a circuit board.

Conductive adhesive gel: This term is used herein to refer to an example of a means of attaching a plurality of electrodes to the skin of an individual in treatment. Conductive adhesive gel can be disposed on top of or around electrical leads to facilitate transfer of the electrical impulse, to secure the electrodes to the individual's body, and to prevent direct contact between the individual's skin and the electrical leads.

Contact portion: This term is used herein to refer to the aspect of the electrode that contacts and attaches to the skin of the individual in treatment. Electrical impulses are transmitted through the contact portion to the individual.

Controller component: This term is used herein to refer to an electronic part on a circuit board that manages the overall operation of the current device, including the pulses and BLUETOOTH communication. The controller component generates and transmits stimulating signals and pulse width modulated signals to the stimulation component, while receiving variation potential signals from the stimulator component.

Electrical lead: This term is used herein to refer to an electrical connection including a length of wire or metal (e.g., copper) pad or contact on a device. The electrical leads herein are used to transmit the outgoing electrical signal as an electrical impulse, from the circuit board to individual.

Electrode: This term is used herein to refer to a terminal through which electric current passes from a metallic part of a circuit to the nonmetallic part of the circuit (e.g., individual's body). An electrode can collect or emit electric energy within the overall semiconducting system. As used herein, electrodes are attached to the outer skin of an individual in treatment. Electrical impulses can then be transmitted through the electrodes to the individual.

Electronic device: This term is used herein to refer to a system that is capable of wirelessly (e.g., BLUETOOTH, WI-FI, NFC, etc) monitoring and controlling the output or electrical signals discharged by the electrodes. Examples of electronic devices include, but are not limited to, smartphones, dedicated devices, computers, and tablets.

Electrostimulation: This term is used herein to refer to the elicitation of muscle contraction using electrical impulses. Electrostimulation may be used, for example, in particular positions on an individual's body to alleviate pain.

High voltage charge: This term is used herein to refer to an electrical impulse having a voltage that is relatively higher than the moment immediately prior to that electrical impulse.

Housing relation: This term is used herein to refer to a spatial relationship between two items, such that second item is disposed around a portion of the first item in a manner that allows the first item to be covered. For example, conducting adhesive gel is disposed in housing relation to electrical leads that are attached to the exterior of an electrode. Thus, part of the electrical lead is directly attached to and is covered by the electrode. The exposed portion of the lead, therefore, is covered by the adhesive gel.

Incoming electrical signal: This term is used herein to refer to a function that conveys or transmits information or data from the electronic device to the electrode (i.e., components on the circuit board).

Interval: This term is used herein to refer to a cycle or period of time from the beginning of an electrical burst or electrical pulse to the beginning of the immediately subsequent burst or pulse with the rest or lower voltage period therebetween.

Low voltage signal: This term is used herein to refer to a function that conveys or transmits information or data between two entities or components at a relatively low voltage level. For example, low voltage signals can be passed between two components (e.g., controller component and stimulator component) mounted on a circuit board to transmit information or data between the two components to monitor and control the electrical signals received and outputted.

Master component: This term is used herein to refer to a device or process, for example a smartphone with associated software application, that has unidirectional control over another device or process, for example electrodes or electronic processes contained therein.

Non-contact portion: This term is used herein to refer to the aspect of the electrode that does not contact the skin of the individual in treatment.

Outgoing electrical signal: This term is used herein to refer to a function that conveys or transmits information or data (e.g., in the form of an impulse) from the electrode to the electrical leads on the opposite planar side of the circuit board.

Pulse width modulated signal: This term is used herein to refer to a function that conveys or transmits information or data between two components (e.g., controller component and stimulator component), said information or data including a fixed-width high (on-state) component and a variable with low (off-state) component, used within the charge pump circuit of the electrode. As used herein, the controller component generates the pulse width modulated signal and transmits it to the stimulator component to indicate the on- and off-states within the electrical impulses and bursts.

Pulse: This term is used herein to refer to a temporary high voltage charge, a plurality of which makes up a burst. Each pulse has a predetermined strength (i.e., voltage), length/width, and interval.

Slave component: This term is used herein to refer to a device or process, for example electrodes or electronic processes contained therein, that are subject to unidirectional control from another device or process, for example a smartphone with associated software application. The slave component reacts to the signals of the master component.

Software application: This term is used herein to refer to a computer-implemented methodology accessible from a non-transitory, computer-readable media and providing instructions for a computer processor to monitor and control the incoming and outgoing electrical signals, including parameters and settings thereof, relative to the electrodes and the circuit boards contained therein.

Stimulating signal: This term is used herein to refer to a function that conveys or transmits information or data between two components (e.g., controller component and stimulator component), said information or data indicating the need for a charge to be delivered to the electrodes. As used herein, the controller component generates the stimulating signal and transmits it to the stimulator component to indicate need for electrostimulation.

Stimulation signal: This term is used herein to refer to a pulse of electric current that is typically transmitted through an electrode onto an individual's body. The term “stimulation signal” is synonymous with electrical impulses.

Stimulator component: This term is used herein to refer to an electronic part on a circuit board that generates the high voltage charge that is delivered as electrical pulses to electrodes that are in contact with the individual in treatment. The stimulator component generates and transmits variation potential signals to the controller component, while receiving stimulating signals and pulse width modulated signals from the controller component.

Treatment: This term is used herein to refer to the process or manner of remedying an issue experienced by an individual.

Variation potential signal: This term is used herein to refer to a function that conveys or transmits information or data from the stimulator charge-pump capacitor to the controller component, said information or data including a measurement of voltage as determined in the predetermined voltage level versus actual output of voltage. If variation potential voltage is lower than as previously set, the pulse width modulated waveform is increased in frequency (i.e., shorter low-states) to speed up the charge pump and raise the voltage. Thus, the controller performs a closed-loop to control the charge pump with output pulse width modulated signal and feedback variation potential to achieve a regulated voltage.

Waveform: This term is used herein to refer to the shape and form of an electrical pulse. Examples of waveforms include, but are not limited to, a sine wave, a square wave, a triangle wave, a sawtooth wave, and other composite and periodic waveforms. Waveforms of the electrical pulses discussed herein are adjustable prior to, during, or after a treatment by an electronic device.

Wireless: This term is used herein to refer to communication between two items without a wired connection. Examples of wireless connections include, for example, BLUETOOTH, WI-FI radio waves, near field communication, etc.

The advantages set forth above, and those made apparent from the foregoing disclosure, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A wireless electrostimulation system for generating and controlling stimulation signals delivered to the skin of an individual, comprising:
at least one electrode including a housing having a contact portion and a non-contact portion forming a closed hollow interior therein, said contact portion being substantially planar and having an inner surface and an outer surface;
a circuit board disposed on said inner surface of said contact portion, said circuit board having a battery and a plurality of components that receive an incoming electrical signal containing data and transmit an outgoing electrical signal in the form of an electrical impulse on said skin of said individual;
said plurality of components including a controller component that manages operation of said plurality of components;
said plurality of components further including a stimulator component that generates a high voltage charge that is delivered as said electrical impulses;
said electrical impulses formed of a plurality of bursts, each of said plurality of bursts formed of a plurality of pulses corresponding to said high voltage charge;
said each burst having a predetermined interval and each of said plurality of pulses having a predetermined waveform, a predetermined voltage level, and a predetermined interval, collectively forming parameters included in said data in said incoming electrical signal received by said controller component;
said plurality of components further including a plurality of low voltage signals transmitted between said controller component and said stimulator component to monitor and control said outgoing electrical signal, said plurality of low voltage signals including one or more signals selected from the group consisting of a stimulating signal, a pulse width modulated signal, and a variation potential signal,
said stimulating signal generated by said controller component and providing notification to said stimulator component of when said outgoing electrical signal is to be delivered to said electrode from said stimulator component,
said pulse width modulated signal generated by said controller component and providing increase and decrease of voltage within said each burst,
said variation potential signal generated by said stimulator component and providing notification to said controller component to increase or decrease output of said pulse width modulated signal;
one or more electrical leads disposed on said outer surface of said contact portion;
a conductive adhesive gel disposed in housing relation to each of said one or more electrical leads to adhere to said skin of said individual, said outgoing electrical signal outputted from said circuit board and transmitted to said skin of said individual through said each electrical lead and said conductive adhesive gel; and
an electronic device configured to wirelessly transmit said incoming electrical signal from said electronic device to said at least one electrode.

2. A wireless electrostimulation system as in claim 1, further comprising:
an activation button positioned within said closed hollow interior of said housing for activating said at least one electrode when depressed.

3. A wireless electrostimulation system as in claim 1, further comprising:
said parameters following an acupuncture protocol.

4. A wireless electrostimulation system as in claim 1, further comprising:
said electronic device including a master component;
said plurality of components within said hollow interior of said housing including a slave component;
said master component and said slave component being in wireless, electrical communication with each other to monitor and control said outgoing electrical signal.

5. A wireless electrostimulation system as in claim 4, further comprising:
said wireless, electrical communication being a BLUETOOTH connection between said master component and said slave component.

6. A method for controlling delivery of stimulation signals to an individual in need, comprising the steps of:
providing a plurality of electrodes each having a housing, a circuit board with components imbedded within an interior of said housing, one or more electrical leads on the exterior of said housing, and conducting adhesive gel disposed in overlying relation to said one or more electrical leads,
said each electrode capable of receiving an incoming electrical signal and transmitting an outgoing electrical signal that is associated with an electrical impulse delivered to said individual, said electrical impulse formed of a plurality of bursts. said bursts formed of a plurality of pulses;
providing an electronic device that can monitor and control said plurality of electrodes;
evaluating said individual to determine a treatment needed;
determining a treatment protocol for said individual based on said treatment needed by said individual;
wirelessly transmitting said incoming electrical signal to said circuit board from said electronic device, said incoming electrical signal including said treatment protocol that contains one or more parameters corresponding to said electrical impulse, said one or more parameters selected from the group consisting of waveform of each of said plurality of pulses, voltage level of said each pulse, interval of said each pulse, and interval of each of said plurality of bursts,
said components including a controller component and a stimulator component in electrical communication with each other, said electrical communication including one or more low voltage signals selected from the group consisting of a stimulating signal, a pulse width modulated signal, and a variation potential signal, said stimulating signal generated by said controller component and providing notification to said stimulator component of when said outgoing electrical signal is to be delivered to said electrode from said stimulator component, said pulse width modulated signal generated by said controller component and providing increase and decrease of voltage within said each burst, said variation potential signal generated by said stimulator component and providing notification to said controller component to increase or decrease output of said pulse width modulated signal;
attaching said plurality of electrodes to an exterior skin surface of said individual by contacting said skin surface with said conducting adhesive gel to permit contact between said skin surface and said one or more electrical leads;
activating said each electrode to initiate said treatment, transmit said electrical impulse to said individual;
monitoring and controlling said each burst and said each pulse through said electronic device.

7. A method for controlling delivery of stimulation signals as in claim 6, further comprising:
said steps of determining said treatment protocol and wirelessly transmitting said incoming electrical signal achieved automatically by a software application installed on said electronic device upon selection of said treatment needed by said individual.

8. A method for controlling delivery of stimulation signals as in claim 6, further comprising:
positioning a push activation button within said hollow interior of said housing;
said step of activating said each electrode performed by depressing the exterior of said housing to depress said push activation button.

9. A method for controlling delivery of stimulation signals as in claim 6, further comprising:
said treatment protocol corresponding to an acupuncture protocol.

10. A method for controlling delivery of stimulation signals as in claim 6, further comprising:
said electronic device including a master component;
said components within said interior of said housing including a slave component;
said master component and said slave component being in wireless, electrical communication with each other to monitor and control said outgoing electrical signal.

11. A method for controlling delivery of stimulation signals as in claim 10, further comprising:
said step of wirelessly transmitting said incoming electrical signal to said circuit board performed via a BLUETOOTH connection between said master component and said slave component.

12. An electrode system for generating and delivering stimulation signals to an individual in need, comprising:
a circuit board containing a controller component and a stimulator component;
a flexible housing snugly fitted in overlying relation to said circuit board to form a hollow interior therein, said controller component and said stimulator component secured within said hollow interior, said controller component and said stimulator component both in communication with an electrical device external to said flexible housing;
a push button positioned on said circuit board and in electrical communication with said stimulator component to activate transmission of said stimulation signals to said individual;
a battery disposed in overlying relation to said push button, whereby when said housing is depressed, a force is applied to said battery causing said push button to be depressed, thus activating said electrode system;
a plurality of copper contacts attached to and disposed in underlying relation to said circuit board; and
conductive adhesive gel disposed in housing relation to each of said plurality of copper contacts, said gel conducting said stimulation signals from said circuit board to said individual.
